(19) United States
(12) Patent Application Publication
(10) Pub. No.: US 2025/0256033 A1
(43) Pub. Date: Aug. 14, 2025

(54) Syringe for Medicament Cartridges Having Varying Sizes

(71) Applicant: Becton, Dickinson and Company, Franklin Lakes, NJ (US)

(72) Inventors: Nandha Krishnan, Coimbatore (IN); Kowshika K, Tiruppur (IN); Sabarinathan Rajendran, Krishnagiri (IN); Hariharan Iyer, Clifton, NJ (US); Tirtharup Bhattacharjee, Ahmedabad (IN)

(21) Appl. No.: 18/859,184

(22) PCT Filed: Apr. 28, 2023
(86) PCT No.: PCT/US23/20298

§ 371 Date: Oct. 23, 2024

(30) Foreign Application Priority Data
Apr. 29, 2022 (IN) 202211025346

(51) Int. Cl. A61M 5/24; A61M 5/315; A61M 5/34

(52) U.S. Cl. CPC A61M 5/2422; A61M 5/2459; A61M 5/31551; A61M 5/34; A61M 2005/2462

(57) ABSTRACT

A syringe for dispensing medicament from a cartridge, the syringe including a housing having a proximal end, a distal end, and a sidewall defining a passageway adapted to receive the cartridge, an adjustment system comprising an adjustment knob positioned at the proximal end of the housing and two or more adjustment plates extending into the passageway of the housing, and a plunger comprising a plunger rod, the plunger rod extending though a through-hole in the adjustment knob and into the passageway of the housing, wherein rotation of the adjustment knob with respect to the housing causes a distance between the adjustment plates to be changed and a distance between the adjustment plates and an interior of the sidewall of the housing to be changed. The syringe may also include a needle assembly.

### CROSS-REFERENCE TO RELATED APPLICATION

The present application claims priority to Indian Application No. 202211025346, filed Apr. 29, 2022, entitled “Syringe for Medicament Cartridges Having Varying Sizes”, the entire disclosure of which is hereby incorporated by reference in its entirety.

### BACKGROUND OF THE INVENTION

### Field of the Invention

The present invention is directed to a syringe for dispensing medicament from a cartridge, and more specifically, to a syringe for dispensing medicament from cartridges having different sizes.

### Description of Related Art

The provision of medicaments in cartridges that can be used in combination with reusable syringes provides a convenient way of storing and dispensing the medicaments to patients. Such prior art reusable syringes are designed to hold a cartridge having a specific diameter. Such syringes are described in U.S. Pat. Nos. 2,475,939; 3,462,840; and 5,122,057, and European Patent No. EP 1225933. Therefore, different syringes are needed depending on the size of the cartridge that is to be used.

Medicaments, such as biologics, are being required to be administered at higher injection volumes and viscosities than in the past. Therefore, there is a need to provide a convenient packaging and delivery method for delivering these medicaments.

Thus, there is a need for a universal reusable syringe that can be utilized with cartridges having a variety of different diameters and lengths.

### SUMMARY OF THE INVENTION

The present invention is directed to a syringe for dispensing medicament from a cartridge. The syringe comprises a housing having a proximal end, a distal end, and a sidewall defining a passageway adapted to receive the cartridge, an adjustment system comprising an adjustment knob positioned at the proximal end of the housing and two or more adjustment plates extending into the passageway of the housing, and a plunger comprising a plunger rod, the plunger rod extending though a through-hole in the adjustment knob and into the passageway of the housing. Rotation of the adjustment knob with respect to the housing causes a distance between the adjustment plates to be changed and a distance between the adjustment plates and an interior of the sidewall of the housing to be changed.

A bottom surface may extend inwardly from the sidewall at the distal end of the housing, an opening may be provided in the bottom surface, and a collar adapted to engage a needle hub of a needle assembly may extend around the opening. At least one opening is provided in the sidewall of the housing through which the cartridge can be placed in the passageway. A flange may extend from the proximal end of the housing.

The plunger is movable with respect to the housing such that a distal force applied to the plunger while holding the housing stationary causes the plunger to be displaced in a distal direction within the passageway of the housing. The plunger further comprises an engagement portion adapted to engage a stopper of the cartridge extending from a distal end of the plunger rod and/or a thumb pad extending from a proximal end of the plunger rod. The engagement portion of the plunger may be stepped, comprising portions having different dimensions which correspond to the dimensions of a variety of stoppers used in various cartridges.

The adjustment plates may be elongated plates that extend in a longitudinal direction from the proximal end of the housing to the distal end of the housing. The adjustment plates may have a curvature corresponding to an outer curvature of the cartridge allowing each plate to partially surround the outer surface of the cartridge when the cartridge is contained in the passageway of the housing. The adjustment plates may be positioned with respect to one another at equal increments around a circumference of the passageway of the housing. When two adjustment plates are provided, the adjustment plates may be positioned substantially opposite one another. The syringe may have an even number of adjustment plates and a corresponding number of camming portions.

The adjustment knob comprises an engagement portion that extends into the passageway of the housing and a grip portion that extends above the proximal end of the housing. Proximal ends of the adjustment plates extend into the engagement portion of the adjustment knob, and distal ends of the adjustment plates are adjacent the distal end of the housing. The engagement portion of the adjustment knob comprises two or more camming portions, each camming portion having a first end, a second end, a distal surface, and a sidewall, wherein a radial length of the distal surface of each camming portion increases from the first end to the second end, such that a radial length at the first end of each camming portion is smaller a radial length at the second end of the camming portion. The first end of a first camming surface may be adjacent the second end of a second camming portion. The sidewalls of the camming portions define an opening having a maximum width between the first ends of the camming portions and a minimum width between the second ends of the camming portions. The syringe may have two adjustment plates and two camming portions where each camming portion extends 180° around a circumference of the engagement portion of the adjustment knob.

The sidewalls of the camming portions and the plunger rod may define a slot having a radial width increasing from the second end of each camming portion to the first end of the camming portion. Proximal ends of the adjustment plates are received in the slot such that each adjustment plate is associated with a corresponding camming portion.

With the adjustment knob rotated to a position in which the adjustment plates are each received in the slot adjacent a corresponding first end of a camming portion, the adjustment plates are at a maximum distance from one another; with the adjustment knob rotated to a position in which the adjustment plates are each received in the slot adjacent a corresponding second end of a camming portion, the adjustment plates are at a minimum distance from one another, and with the adjustment knob rotated to a position in which the adjustment plates are each received in the slot at a position between the first end of a camming portion and the second end of the camming portion, the adjustment plates are at distance from one another that is between the maximum distance and the minimum distance.

An indicator may be provided on the adjustment knob and indicia may be provided on a flange extending from the proximal end of the housing, where the indicator in combination with the indicia indicate the position of the adjustment plates.

The syringe may further comprise a needle assembly comprising a needle having a beveled point at each end and a needle holder comprising a needle hub, wherein the housing further comprises a bottom surface extending inwardly from the sidewall at the distal end of the housing, an opening in the bottom surface, and a collar extending around the opening and adapted to engage the needle hub. The needle passes through a through hole in the needle holder, such that a portion of the needle extends proximally from the needle holder, and a portion of the needle extends distally from the needle holder. When the needle hub of the needle holder is inserted into the opening in the collar of the housing, the portion of the needle extending proximally from the needle holder passes through the opening in the bottom surface of the housing and into the passageway, and when the container is contained in the passageway, the portion of the needle extending proximally from the needle holder pierces a pierceable septum of the cartridge creating a fluid path for medicament to pass from the cartridge through the needle.

A locking assembly may be provided for removably locking the needle hub within the collar of the housing. The locking mechanism may comprise at least one flexible finger extending from an outer surface of the needle hub and a corresponding recess in the opening in the collar of the housing. The flexible finger may comprise a first portion attached to the hub and a second portion extending in the longitudinal direction from the first portion, the recess may comprise a first portion and a second portion extending in a transverse direction from the first portion of the recess, and a width of the first portion of the recess in a lateral direction may correspond to a width of the first portion of the flexible finger in the lateral direction and a width of the second portion of the flexible finger in the lateral direction corresponds to a width of the second portion of the flexible finger in the lateral direction. With the needle hub inserted into the opening of the collar of the housing, the first portion of the flexible finger abuts an interior end of the second portion of the recess and locks the needle holder in the opening in the collar. Exertion of a biasing force on a portion of the second portion of the flexible finger extending from the second portion of the recess releases the engagement between the interior end of the second portion of the recess and the first portion of the flexible finger and unlocks the needle holder from the opening in the collar.

### BRIEF DESCRIPTION OF THE DRAWINGS

FIG. 1 is a side perspective view of a syringe including a needle assembly according to the invention.

FIG. 2 is a cross-sectional view of a syringe including a needle assembly according to the invention prior to injection of the medicament into a patient.

FIG. 3 is a cross-sectional view of a syringe including a needle assembly according to the invention during injection of the medicament into a patient.

FIG. 4 is a side perspective view of the adjustment knob of a syringe according to the invention.

FIG. 5 is a cross-sectional view of a syringe according to the invention showing the adjustment knob and adjustment plates in a first position.

FIG. 6 is a cross-sectional view of a syringe according to the invention showing the adjustment knob and adjustment plates in a second position.

FIG. 7 is a proximal end view of a syringe according to the invention.

FIG. 8 is an alternative plunger rod for a syringe according to the invention.

FIG. 9 is an exploded side perspective view of a syringe according to the invention.

FIG. 10 is an exploded side perspective view of a needle assembly for a syringe according to the invention.

FIG. 11 is a side perspective view of a syringe according to the invention, a needle assembly for the syringe and a cartridge used with the syringe.

FIG. 12 is a side perspective view of a syringe without a needle assembly according to the invention.

FIG. 13A is a cross-sectional view of a syringe according to the invention prior to use.

FIG. 13B is a cross-sectional view of a syringe according to the invention after a cartridge of medicament has been inserted into the syringe.

FIG. 13C is a cross-sectional view of a syringe according to the invention after the adjustment knob has been rotated to bring the adjustment plates into contact with the sidewall of the cartridge.

FIG. 13D is a cross-sectional view of a syringe according to the invention after the cartridge has been fixed in the syringe by the adjustment plates and the engagement portion of the plunger rod has been brought into contact with the stopper of the cartridge.

FIG. 13E is a cross-sectional view of a syringe according to the invention after the needle assembly has been attached.

### DESCRIPTION OF THE INVENTION

As used herein, unless otherwise expressly specified, plural encompasses singular and vice versa. When ranges are given, any endpoints of those ranges and/or numbers within those ranges can be combined with the scope of the present invention. “Including”, “such as”, “for example” and like terms means “including/such as/for example but not limited to”.

For purposes of the description hereinafter, spatial orientation terms, as used, shall relate to the referenced embodiment as it is oriented in the accompanying drawings, figures, or otherwise described in the following detailed description. However, it is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

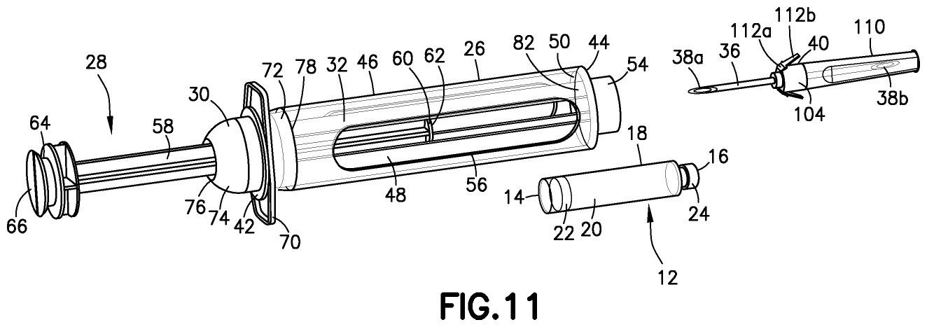

The present invention is directed to a universal syringe 10 that is adapted to accommodate a variety of cartridges 12 for a medicament having different diameters and/or lengths. As shown in FIG. 11, such cartridges comprise a proximal end 14, a distal end 16, and a sidewall 18 extending from the proximal end 14 to the distal end 16 and defining a chamber 20 adapted to contain a medicament. The proximal end 14 is closed by a stopper 22, and the distal end 16 is closed by a pierceable septum 24. The application of a distal force to the stopper 22, moves the stopper 22 from a first position within the chamber 20 adjacent the proximal end 14 of the cartridge 12 to a second position within the chamber 20 adjacent the distal end 16 of the cartridge 12, thereby dispensing the medicament through a needle inserted through the pierceable septum 24 at the distal end 16 of the cartridge 12.

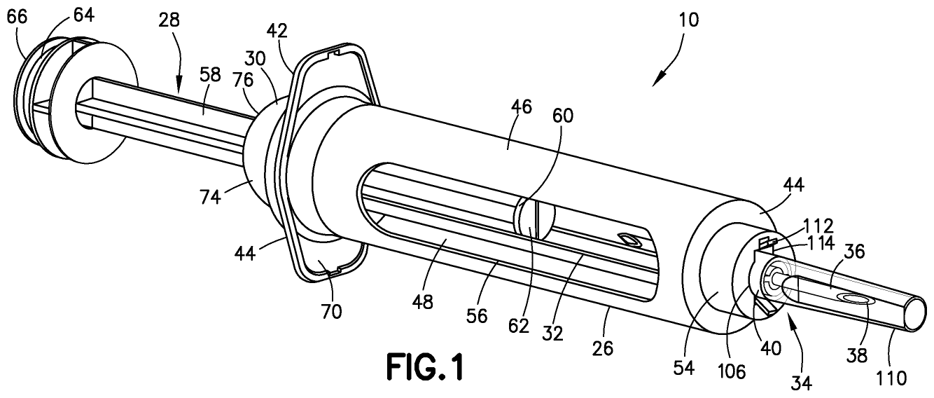

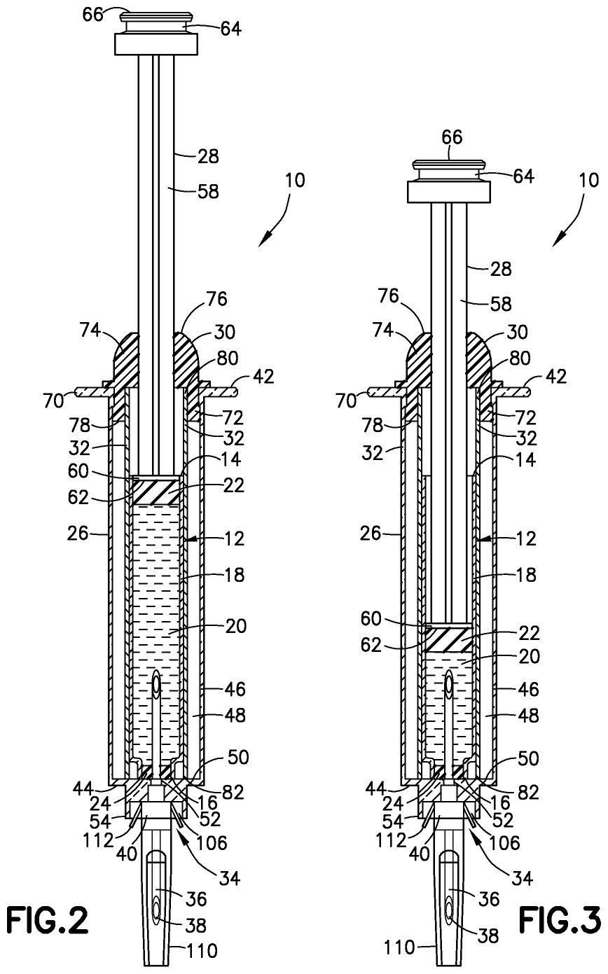

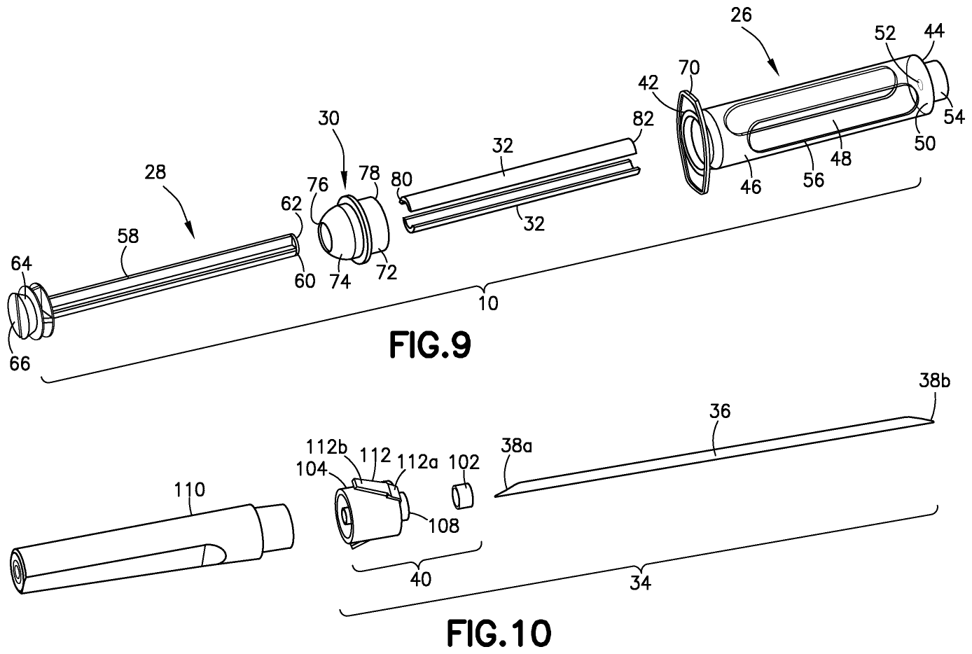

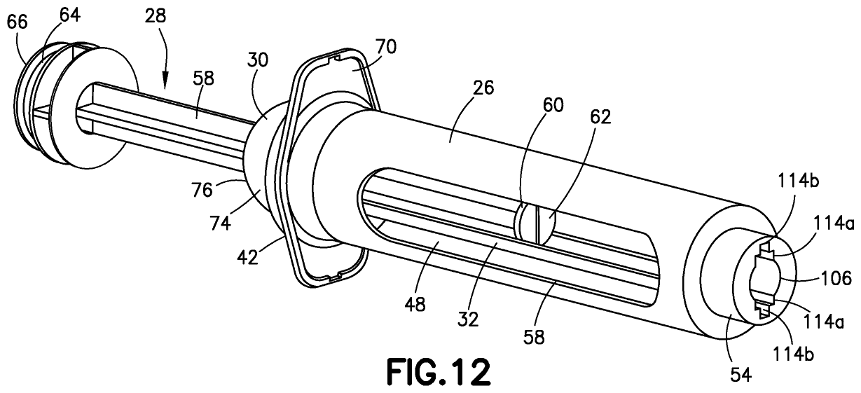

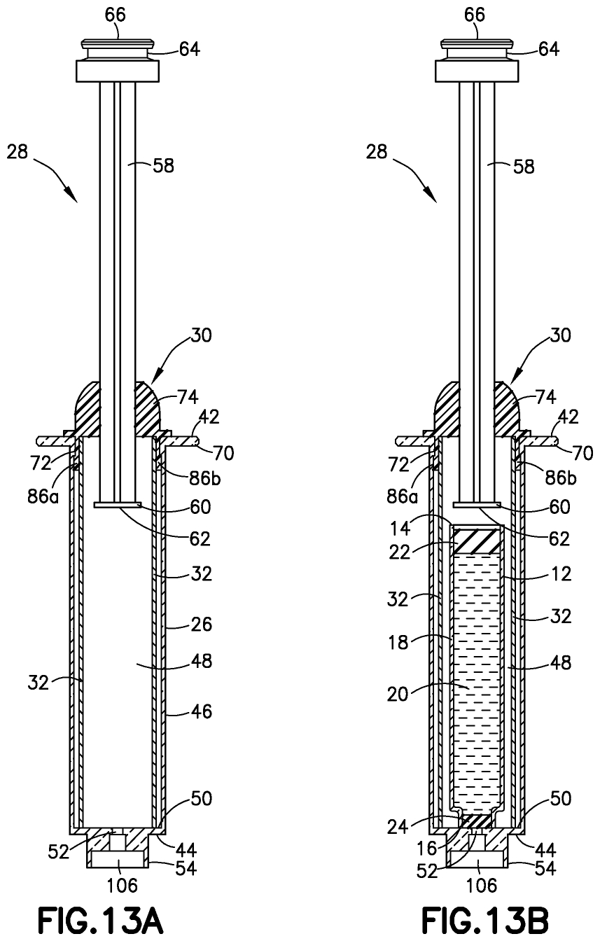

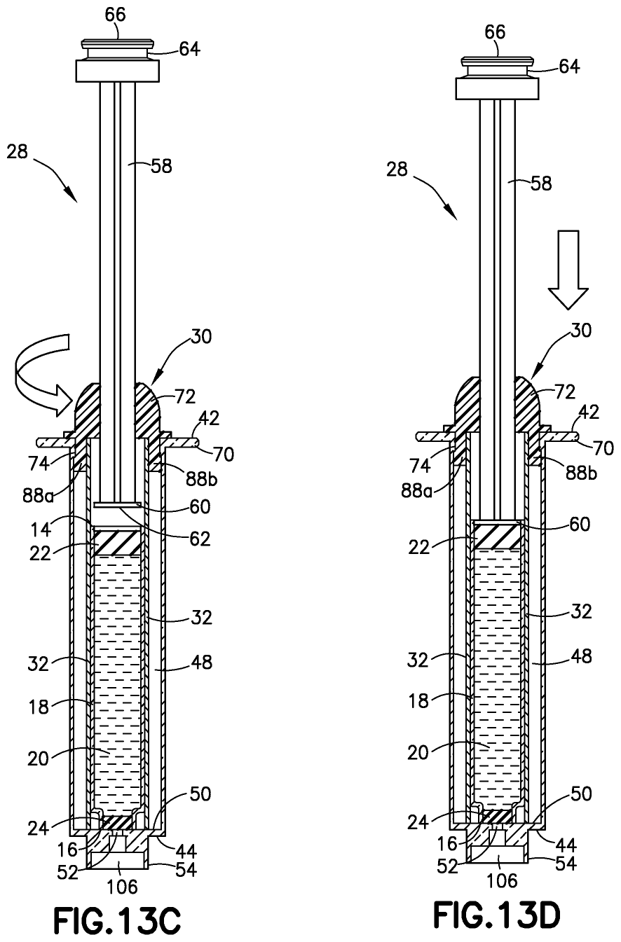

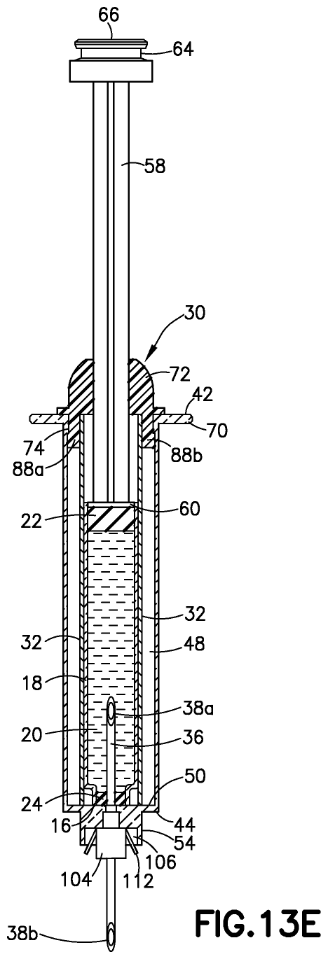

As shown in FIGS. 1-3, 9, 11, and 12, the syringe 10 comprises a housing 26, a plunger 28, and an adjustment system comprising an adjustment knob 30 and two or more adjustment plates 32. A needle assembly 34 comprising a needle 36 having a beveled point 38a, 38b at each end and needle holder 40 may be provided for attachment to the housing 26 (FIGS. 10-12).

The housing 26 has a proximal end 42, a distal end 44, and a sidewall 46 extending between the proximal end 42 and distal end 44. The sidewall 46 defines a passageway 48 adapted to receive a medicament cartridge 12. The passageway 48 may be substantially cylindrical. A bottom surface 50 extends inwardly from the sidewall 46 at the distal end 44 of the housing 26. An opening 52 is provided in the bottom surface 50 and a collar 54 extends distally from the bottom surface 50 and surrounds the opening 52. The collar 54 is adapted to engage the needle holder 40 of a needle assembly 34.

At least one opening 56 is provided in the sidewall 46 of the housing 26 through which the cartridge 12 can be placed in the passageway 48. The opening 56 may have a length extending along the sidewall 46 in a longitudinal direction L extending from the proximal end 42 to the distal end 44 and a width extending around a circumference of the sidewall 46 in a direction perpendicular to the length of the opening 56. The length and width of the opening 56 are sufficient to allow a cartridge 12 having the largest cross-section and length that the syringe 10 is intended to accommodate to be inserted through the opening 56.

The plunger 28 comprises a plunger rod 58, an engagement portion 60 extending from a distal end 62 of the plunger rod 58, and a thumb pad 64 extending from a proximal end 66 of the plunger rod 58 in a direction perpendicular to the plunger rod 58. The engagement portion 60 of the plunger 28 is contained within the passageway 48 of the housing 26 and adapted to engage the stopper 22 of the cartridge 12. The plunger 28 is movable with respect to the housing 26 such that a distal force applied to the plunger 28 while holding the housing 26 stationary causes the engagement portion 60 of the plunger 28 to be displaced in a distal direction within the passageway 48 of the housing 26. The engagement portion 60 of the plunger 28 may be stepped (FIG. 8), comprising portions 68 having different dimensions which correspond to the dimensions of a variety of stoppers 22 used in various cartridges 12.

A flange 70 may extend from the proximal end 42 of the housing 26. The flange 70 is adapted to accommodate a user's fingers to apply a counterforce when the user is using their thumb to apply a distal force to the plunger 28 to administer the medicament to a patient.

An alignment system comprising an adjustment knob 30 provided at a proximal end 42 of the housing 26 and two or more adjustment plates 32 extending into the passageway 48 of the housing 26.

The adjustment knob 30 is positioned at the proximal end 42 of the housing 26 and comprises a distal engagement portion 72 that extends into the passageway 48 and a proximal grip portion 74 that extends above the proximal end 42 of the housing 26. A through-hole extends from the proximal end 76 of the adjustment knob 30 to the distal end 78 of the adjustment knob 30. The plunger rod 58 of the plunger 28 passes through the through-hole in the adjustment knob 30.

Two or more adjustment plates 32 are provided to align and hold the cartridge 12 within the passageway 48 of the housing 26. The adjustment plates 32 are elongated plates that extend in the passageway 48 in the longitudinal direction from the proximal end 42 of the housing 26 to the distal end 44 of the housing 26. The adjustment plates 32 may have a curvature corresponding to the curvature of an outer surface of the cartridge 12 allowing each adjustment plate 32 to partially surround the outer surface of the cartridge 12 when the cartridge 12 is contained in the passageway 48 of the housing 26. The adjustment plates 32 may be positioned with respect to one another at equal increments around the circumference of the passageway 48 of the housing 26. For example, when two adjustment plates 32a, 32b are provided, the adjustment plates 32a, 32b are positioned substantially opposite one another. If four adjustment plates were provided, the adjustment plates would be positioned at 90° angles to one another. The proximal ends 80 of the adjustment plates 32 extend into the engagement portion 72 of the adjustment knob 30, and the distal ends 82 of the adjustment plates 32 abut the bottom surface 50 of the housing 26.

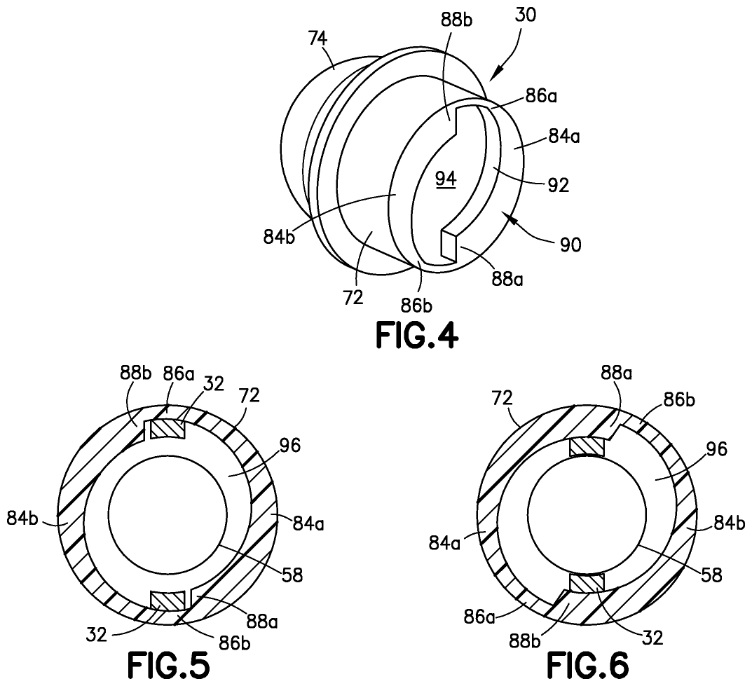

As shown in FIGS. 4-6, the engagement portion 72 of the adjustment knob 30 comprises two or more camming portions 84 extending in the longitudinal direction. Each camming portion 84 has a first end 86, a second end 88, a distal surface 90, and a sidewall 92. The radial length of the distal surface 90 of each camming portion 84 increases from the first end 86 to the second end 88, such that the radial length at the first end 86 of each camming portion 84 is smaller than the radial length at the second end 88 of the camming portion 84. The radial length of the distal surface 90 of each camming portion 84 may increase in a continuous manner from the first end 86 to the second end 88. When two camming portions 84 are provided, each camming portion 84 extends 180° around the circumference of the engagement portion 72 of the adjustment knob 30. The first end 86a of the first camming surface 84a is adjacent the second end 88b of the second camming portion 84b, such that the first end 86a of the first camming portion 84a is substantially opposite the first end 86b of the second camming portion 84b. The sidewalls 92 of the camming portions 84 define an opening 94 having a maximum width between the first ends 86a, 86b of the camming portions 84a, 84b and a minimum width between the second ends 88a, 88b of the camming portions 84a, 84b.

The adjustment knob 30 is rotatable with respect to the housing 26.

With the plunger rod 58 extending through the through-hole of the adjustment knob 30, the sidewalls 92 of the camming portions 84 and the plunger rod 58 define a slot 96 having a radial width increasing from the second end 88 of each camming portion 84 to the first end 86 of the camming portion 84. The proximal ends of the adjustment plates 32 are received in the slot 96 such that each adjustment plate 32 is associated with a corresponding camming portion 84.

The adjustment knob 30 may be rotated with respect to the housing 26 and the adjustment plates 32 by grasping and turning the grip portion 74 of the adjustment knob 30. By rotating the adjustment knob 30 with respect to the housing 26, the distance between the adjustment plates 32 and correspondingly the distance between the adjustment plates 32 and an interior of the sidewall 46 of the housing 26 can be changed.

With the adjustment knob 30 rotated to a position in which the adjustment plates 32a, 32b are each received in the slot 96 adjacent a corresponding first end 86a, 86b of a camming portion 84a, 84b, the adjustment plates 32a, 32b are at a maximum distance D₁ from one another. With the adjustment knob 30 rotated to a position in which the proximal ends 80 of the adjustment plates 32a, 32b are each received in the slot 96 adjacent a corresponding second end 88a, 88b of a camming portion 84a, 84b, the adjustment plates 32a, 32b are at a minimum distance D₂ from one another. With the adjustment knob 30 rotated to a position in which the adjustment plates 32a, 32b are each received in the slot 96 at a position between the first end 86a, 86b of a camming portion 84a, 84b and the second end 88a, 88b of a camming portion 84a, 84b, the adjustment plates 32a, 32b are at distance from one another that is between the maximum distance D₁ and the minimum distance D₂.

As the adjustment knob 30 is rotated, at the position of each of the adjustment plates 32 in the slot 96, the radial distance of the distal surface 90 of the camming portions 84 is increased or decreased, thereby causing the sidewalls 92 of the camming portions 84 to force the adjustment plates 32 radially inward or allow the adjustment plates 32 to move radially outward, respectively. As a result of the movement of the adjustment plates 32 by the camming portions 84 of the adjustment knob 30, the distance between the adjustment plates 32 and correspondingly the distance between the adjustment plates 32 and an interior of the sidewall 46 of the housing 26 can be changed.

With a cartridge 12 inserted in the passageway 48 of the housing 26, the adjustment plates 32 can be brought into contact with the outer surface of the cartridge 12 by rotating the adjustment knob 30, thereby supporting the cartridge 12 within the passageway 48 and aligning the pierceable septum 24 of the cartridge 12 with the opening 52 in the bottom surface 50 of the housing 26.

In addition to the interaction between the adjustment plates 32a, 32b and the slot 96, optional slots or guide plates extending in the longitudinal direction of the interior surface of the sidewall 46 of the housing 26 and/or slots or guide plates extending in the radial direction along the interior surface of the bottom surface 50 of the housing 26 may be included to aid in the positioning of the adjustment plates 32a, 32b relative to one another around the circumference of the housing 26.

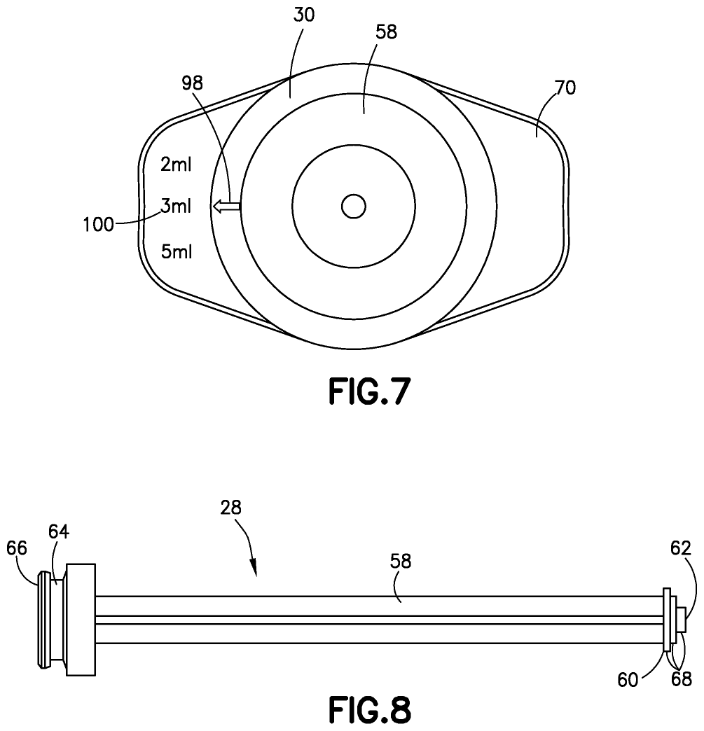

As shown in FIG. 7, an indicator 98, for example, an arrow, may be provided on the adjustment knob 30 and indicia 100 may be provided on the flange 70 of the housing 26. As the adjustment knob 30 is rotated, the indicator 98 corresponds to the indicium 100 indicating the position of the adjustment plates 32. The indicia 100 may correlate to the placement of the adjustment plates 32 to specific size cartridges holding different amount of medicament, for example, 2 ml, 3 ml, and 5 ml indicia, which indicate that the adjustment plates 32 are positioned to hold 2 ml, 3 ml, and 5 ml cartridges, respectively.

The syringe 10 may be provided with a needle assembly 34 comprising a needle 36 having a beveled point 38a, 38b at each end and needle holder 40 (FIGS. 10-12). The needle holder 40 comprises a sleeve 102 and a needle hub 104. The sleeve 102 fits securely around the needle 36. The sleeve 102 may be substantially disk-shaped with a through-hole through which the needle 36 passes and may be held around the needle 36 by friction. The needle hub 104 is adapted to be received in a corresponding opening 106 in the collar 54 of the housing 26, has a through-hole through which the needle 36 passes, and includes a recess 108 for receiving the sleeve 102. The sleeve 102 may be frictionally engaged within the recess 108 of the needle hub 104. Alternatively, a single piece needle hub that frictionally engages the needle 36 may be provided. The needle 36 passes through the through-holes in the sleeve 102 and the needle hub 104, such that a portion of the needle 36 extends proximally from the needle holder 40, and a portion of the needle 36 extends distally from the needle holder 40. The needle holder 40 may be positioned substantially midway between the two beveled points 38a, 38b along the shaft of the needle 36.

One or more needle shields 110 covering one or both of the beveled points 38a, 38b of the needle 36 may be provided. A needle shield 110 covering the beveled point 38a that is inserted into the housing 26 of the syringe 10 may be removed prior to the insertion of the needle holder 40 into the collar 54 of the housing 26, and a needle shield covering the beveled point 38b that is inserted into the patient may be removed prior to the insertion of the needle 36 into the patient. In this manner, users are prevented from accidently being stuck with the beveled points of the needle and the needle is kept sterile prior to use.

When the needle hub 104 is inserted into the opening 106 in the collar 54 of the housing 26, the portion of the needle 36 extending proximally from the needle holder 40 passes through the opening 52 in the bottom surface 50 of the housing 26 and into the passageway 48. When a cartridge 12 is contained in the passageway 48, the portion of the needle 36 extending proximally from the needle holder 40 pierces the pierceable septum 24 of the cartridge 12 creating a fluid path for the medicament to pass from the cartridge 12 through the needle 36.

The needle assembly 34 may be removably locked within the collar 54 of the housing 26. The locking assembly for removably locking the needle hub 104 within the collar 54 of the housing 26 may comprise at least one flexible finger 112 extending from an outer surface of the needle hub 104 and a corresponding recess 114 in the opening 106 in the collar 54 of the housing 26. The locking mechanism has a longitudinal direction, which is the direction in which the needle holder 40 is inserted into the opening 106 in the collar 54 of the housing 26, a transverse direction, which is a direction vertically perpendicular to the longitudinal direction, and a lateral direction, which is a direction horizontally perpendicular to the longitudinal direction. The flexible finger 112 comprises a first portion 112a attached to the hub and a second portion 112b extending in the longitudinal direction from the first portion 112a. A width of the first portion 112a of the flexible finger 112 in the lateral direction is greater than a width of the second portion 112b of the flexible finger 112 in the lateral direction. The recess 114 has first portion 114a and a second portion 114b extending in the transverse direction from the first portion 114a. The width of the first portion 114a of the recess 114 in the lateral direction corresponds to the width of the first portion 112a of the flexible finger 112 in the lateral direction and the width of the second portion 114b of the recess 114 in the lateral direction corresponds to the width of the second portion 112b of the flexible finger 112 in the lateral direction.

When the needle holder 40 is inserted longitudinally into the opening 106 of the collar 54 of the housing 26, the first portion 112a of the flexible finger 112 is received in the first portion 114a of the recess 114, and the flexible finger 112 is biased in the transverse direction toward the needle holder 40 by the upper surface of the first portion 114a of the recess 114. As the needle holder 40 is further inserted into the opening 106 of the collar 54 of the housing 26, the first portion 112a of the flexible finger 112 extends past the interior end of the first portion 114a of the recess 114, the biasing force is released from the flexible finger 112, and a portion of the second portion 112b of the flexible finger 112 is received in the second portion 114b of the recess 114. In this positon, the first portion 112a of the flexible finger 112 abuts the interior end of the second portion 114b of the recess 114, and because the first portion 112a of the flexible finger 112 is wider in the lateral direction than the width of the second portion 114b of the recess 114 in the lateral direction, the needle hub 104 is locked in the opening 106 in the collar 54 of the housing 26. By exerting a transverse biasing force on the portion of the second portion 112b of the flexible finger 112 that extends out of the second portion 114b of the recess 114, the engagement between the interior end of the second portion 114b of the recess 114 and the first portion 112a of the flexible finger 112 is released, the first portion 112a of the flexible finger 112 can again enter the first portion 114a of the recess 114, and the needle holder 40 can be pulled from the opening 106 in the collar 54 of the housing 26.

The needle holder 40 and the opening 106 in the collar 54 of the housing 26 can be chosen such that the needle holder 40 can only be inserted in the collar in a single orientation. Alternatively, the locking assembly may be positioned such that the needle holder 40 can only be inserted in the collar in a single orientation. The needle 36 may be fixed in the needle holder in a specific direction, such that, when the needle holder 40 is inserted in the opening 106 in the collar 54 of the housing 26, the orientation of the beveled point 38b of the needle 36 with respect to the syringe 10 is oriented in the desired direction for injection to be made, for example, with the bevel facing up. In this way, the desired orientation of the beveled point 38b of the needle 36 is automatically achieved when the needle assembly 40 is attached to the housing 26.

In use, the plunger 28 of the syringe 10 may be pulled in a proximal longitudinal direction until the engagement portion 60 of the plunger 28 is adjacent the proximal end 42 of the housing 26. A cartridge 12 is then inserted through the opening 56 in the sidewall 46 of the housing 26 and into the passageway 48. The cartridge 12 is positioned such that the distal end 16 of the cartridge 12 having the pierceable septum 24 abuts the bottom surface 50 of the housing 26. The adjustment knob 30 is then turned until the adjustment plates 32 contact and partially surround the outer surface of the cartridge 12. The plunger 28 is then pushed in the distal longitudinal direction until the engagement portion 60 of the plunger 28 contacts the stopper 22 of the cartridge 12. The needle holder 40 of the needle assembly 34 is then inserted into the opening 106 in the collar 54 of the housing 26 with the proximal beveled point 38a of the needle 36 piercing the pierceable septum 24 of the cartridge 12, such that the medicament in the cartridge 12 can pass through the needle 36. The distal beveled end 38b of the needle 36 extending from the distal end 44 of the housing 26 can be used to pierce the skin of a patient, and the plunger 28 can be pushed in a longitudinal distal direction. As the plunger 28 is pushed distally, the engagement portion 60 of the plunger 28 pushes the stopper 22 of the cartridge 12 in a distal direction, thereby forcing the medicament out of the cartridge 12 through the needle 36, and into the patient.

The various components of the syringe 10 and needle assembly 34 can be made from any suitable material including, but not limited to high density polyethylene (HDPE), acrylonitrile butadiene styrene (ABS) or polyoxymethylene (Delrin©).

Cartridges containing 1-10 ml of medicament, having an outer diameter of 5-25 mm or 8-23 mm can be accommodated by the syringe 10.

While the description has focused on a syringe 10 having two adjustment plates 32a, 32b and two corresponding camming portions 84a, 84b, the syringe may have more than two adjustment plates and two corresponding camming portions. By keeping an even number of adjustment plates and an even number of camming portions and having an equal number of adjustment plates and camming portions, it can be assured that all of the adjustment plates will be equally distanced from the sidewall of the housing, thereby aligning and centering the cartridge within the housing.

The inventive syringe allows medicament in cartridges of a variety of diameters and lengths to be administered to the patient with a single syringe. Because the fluid path is through the needle, the medicament is not exposed to any portion of the syringe, thereby allowing the syringe to be multiple uses without sterilization of the syringe between uses while still assuring the sterility of the medicament fluid path. The inventive syringe has a design that is simple with a camming mechanism provided for adjustment rather than a screw mechanism. This makes the inventive syringe easy to manufacture. By providing a separate needle assembly, orientation of the beveled point of the needle for proper introduction into the tissue of the patient can be accomplished. Yet, even with these advantages over the prior art syringes, the inventive syringe operates in a conventional manner that is well-known to users of the prior art syringes.

Whereas particular aspects of this invention have been described above for purposes of illustration, it will be evident to those skilled in the art that numerous variations of the details of the present invention may be made without departing from the invention.

1. A syringe for dispensing medicament from a cartridge, the syringe comprising:
a housing having a proximal end, a distal end, and a sidewall defining a passageway adapted to receive the cartridge;
an adjustment system comprising an adjustment knob positioned at the proximal end of the housing and two or more adjustment plates extending into the passageway of the housing; and
a plunger comprising a plunger rod, the plunger rod extending though a through-hole in the adjustment knob and into the passageway of the housing,
wherein rotation of the adjustment knob with respect to the housing causes a distance between the adjustment plates to be changed and a distance between the adjustment plates and an interior of the sidewall of the housing to be changed.

2. The syringe of claim 1, wherein a bottom surface extends inwardly from the sidewall at the distal end of the housing, an opening is provided in the bottom surface, and a collar adapted to engage a needle hub of a needle assembly extends around the opening.

3. The syringe of claim 1, wherein at least one opening is provided in the sidewall of the housing through which the cartridge can be placed in the passageway.

4. The syringe of claim 1, wherein the plunger is movable with respect to the housing such that a distal force applied to the plunger while holding the housing stationary causes the plunger to be displaced in a distal direction within the passageway of the housing.

5. The syringe of claim 1, wherein the plunger further comprises an engagement portion adapted to engage a stopper of the cartridge extending from a distal end of the plunger rod and/or a thumb pad extending from a proximal end of the plunger rod.

6. The syringe of claim 5, wherein the engagement portion of the plunger may be stepped, comprising portions having different dimensions which correspond to the dimensions of a variety of stoppers used in various cartridges.

7. The syringe of claim 1, further comprising a flange extending from the proximal end to the housing.

8. The syringe of claim 1, wherein the adjustment plates are elongated plates that extend in a longitudinal direction from the proximal end of the housing to the distal end of the housing.

9. The syringe of claim 1, wherein the adjustment plates have a curvature corresponding to an outer curvature of the cartridge allowing each plate to partially surround the outer surface of the cartridge when the cartridge is contained in the passageway of the housing.

10. The syringe of claim 1, wherein the adjustment plates are positioned with respect to one another at equal increments around a circumference of the passageway of the housing.

11. The syringe of claim 1, wherein two adjustment plates are provided and the adjustment plates are positioned substantially opposite one another.

12. The syringe of claim 1, wherein the syringe has an even number of adjustment plates and a corresponding number of camming portions.

13. The syringe of claim 1, wherein the adjustment knob comprises an engagement portion that extends into the passageway of the housing and a grip portion that extends above the proximal end of the housing.

14. The syringe of claim 13, wherein proximal ends of the adjustment plates extend into the engagement portion of the adjustment knob, and distal ends of the adjustment plates are adjacent the distal end of the housing.

15. The syringe of claim 13, wherein the engagement portion of the adjustment knob comprises two or more camming portions, each camming portion having a first end, a second end, a distal surface, and a sidewall, wherein a radial length of the distal surface of each camming portion increases from the first end to the second end, such that a radial length at the first end of each camming portion is smaller a radial length at the second end of the camming portion.

16. The syringe of claim 15, wherein the first end of a first camming surface is adjacent the second end of a second camming portion.

17. The syringe of claim 15, wherein the sidewalls of the camming portions define an opening having a maximum width between the first ends of the camming portions and a minimum width between the second ends of the camming portions.

18. The syringe of claim 15, wherein the syringe comprises two adjustment plates and two camming portions where each camming portion extends 180° around a circumference of the engagement portion of the adjustment knob.

19. The syringe of claim 15, wherein the sidewalls of the camming portions and the plunger rod define a slot having a radial width increasing from the second end of each camming portion to the first end of the camming portion.

20. The syringe of claim 15, wherein distal ends of the adjustment plates are received in the slot such that each adjustment plate is associated with a corresponding camming portion.

21.-30. (canceled)
